(19) United States
(12) Patent Application Publication
(10) Pub. No.: US 2004/0248860 A1
(43) Pub. Date: Dec. 9, 2004

(54) Use of selective progesterone receptor modulators for the treatment of androgen deficiency

(76) Inventor: Kristof Chwalisz, Hawthorn Woods, IL (US)

Correspondence Address: Steven F. Weinstock, Abbott Laboratories, D-377/AP6A, 100 Abbott Park Road, Abbot Park, IL, 60064-6008, US

(21) Appl. No.: 10/714,219

(22) Filed: Nov. 14, 2003

Related U.S. Application Data
(60) Provisional application No. 60/427,625, filed on Nov. 19, 2002.

(51) Int. Cl. A61K 31/56

(52) U.S. Cl. 514/169

(57) ABSTRACT

Methods of increasing testosterone levels using selective progesterone receptor modulators (SPRMs) are provided. SPRMs may therefore be used to treat conditions associated with decreased levels of testosterone such as sexual dysfunction, hypogondism, andropause, muscle wasting, and frailty. Compositions comprising SPRMs are also provided.

This application claims the benefit of U.S. Provisional Patent Application Ser. No. 60/427,625, filed Nov. 19, 2002, the specification of which is hereby incorporated herein by reference.

### TECHNICAL FIELD

The present invention relates to androgens, and more particularly, relates to treating androgen deficiency, and related symptoms, with selective progesterone receptor modulators (“SPRM”).

### BACKGROUND OF THE INVENTION

Testosterone is a steroidal hormone produced by men and women that belongs to a class of hormones known as androgens. Testosterone is a circulating hormone that can circulate freely or, as is typically the case, it can be bound to various proteins. Sex hormone binding globulin (SHBG) is one protein that binds testosterone with such affinity that testosterone bound by SHBG is not biologically active. Testosterone that is not bound by proteins is biologically active and variously referred to as free testosterone.

In men, testosterone is responsible for the development of secondary male sex characteristics such as growth of body hair and sperm maturation. In women, testosterone plays an important role in the development and function of the musculoskelatal and central nervous systems. High levels of SHBG and low levels of free testosterone, and more likely, a combination of the two, may lead to androgen deficiency and the symptoms associated with such deficiency. Women having an androgen deficiency may experience mood changes, loss of energy, persistent and unexplained fatigue, a decrease in well being, as well as frailty and osteoporosis. Men having an androgen deficiency may experience a loss of secondary sex characteristics, muscle and bone loss, frailty and anemia. Studies have also linked biologically active testosterone levels to libido in both men and women.

Sexual dysfunction afflicts both men and women. In one survey, 42% of women complained of one or more sexual difficulties, and 30% of men complained of one or more sexual difficulties. In men, sexual dysfunction typically is associated with erectile dysfunction. In women, sexual dysfunction is most commonly associated with difficulties with arousal, lubrication and orgasm. While such physiological factors are commonly associated with sexual dysfunction, sexual dysfunction includes other attributes that are more psychological in nature. In fact, both men and women having sexual dysfunction have reported problems with a decreased sexual desire, or libido, as well as a lack of responsiveness to sexual stimulation and diminished sexual pleasure.

Attempts to alleviate sexual dysfunction have primarily focused on the physiological aspects of sexual dysfunction. For example, a class of drugs known as PDE5 inhibitors have been indicated for sexual dysfunction. While such compounds have found clinical utility for alleviating the physical symptoms of sexual dysfunction, they typically have little impact on other aspects of sexual dysfunction such as sexual desire or libido. Hence, such existing therapies are helpful with one aspect of sexual dysfunction but are not a panacea.

There is therefore a need for a therapy for increasing biologically active testosterone in order to deal with the broader spectrum of the symptoms associated with sexual dysfunction as well as other conditions associated with low testosterone levels.

### SUMMARY OF THE INVENTION

The present invention provides a) methods of increasing levels of total as well as biologically active testosterone, b) methods of decreasing levels of SHBG, and therefore c) methods of treating symptoms associated with androgen deficiency (and particularly testosterone deficiency) in a patient. According to any of the above methods, the methods generally will comprise administering a therapeutically effective amount of a selective progesterone receptor modulator (SPRM). The methods provided herein may also include administering a drug that is well known for treating more physiological aspects of sexual dysfunction. The methods may also or further include administration of exogenous testosterone.

Kits and dosage forms containing a SPRM and a drug for treating physiological aspects of sexual dysfunction are also provided.

### BRIEF DESCRIPTION OF THE DRAWINGS

FIG. 1 is a graph of testosterone levels as a function of time while on SPRM therapy.

FIG. 2 is a graph of SHBG levels as a function of time while on SPRM therapy.

### DETAILED DESCRIPTION OF THE INVENTION

Surprising new activities for compounds variously referred to as selective progesterone receptor modulators (“SPRMs”) have unexpectedly been discovered. In particular, it has been discovered that administration of SPRMs increases levels of free testosterone by both increasing the levels of total testosterone and decreasing the levels of SHBG. SPRMs therefore increase bioavailable testosterone levels. As a result of such discoveries, methods for increasing free and total testosterone as well as decreasing SHBG levels are provided. Consequently, methods for treating sexual dysfunction are provided. Additionally, methods for treating other conditions that result from androgen deficiency and that may benefit from an increase in free or total testosterone and a concomitant decrease in SHBG levels are therefore provided. Such methods generally comprise administering a therapeutically effective amount of a SPRM to a patient in need of such therapy. In cases of sexual dysfunction, the methods may further comprise administering a therapeutically effective amount of a compound indicated for the physiological aspects of sexual dysfunction, in addition to the SPRM, to a patient in need of such therapy.

SPRMs (“variously referred to as “mesoprogestins”) are a class of progesterone receptor ligands that possess mixed agonistic and antagonistic activity in vivo. SPRMs show a high degree of endometrial selectivity and control of endometrial function without compromising ovarian estrogen production and thus do not induce estrogen deficiency. The antagonistic activity of SPRMs is incomplete inasmuch as SPRMs will not, in effect, completely block progesterone action as observed with progesterone antagonists such as mifepristone (RU486). Hence, SPRMs have incomplete progesterone receptor antagonist activity due to the fact that they also display low levels of agonist activity. From a more quantitative standpoint, SPRMs score lower than progesterone in the McPhail bioassay but higher than compounds such as RU 486 that have, in effect, a complete antagonistic activity of the progesterone receptor. McPhail tests are widely used to semiquantitatively assess agonistic and antagonistic effects of compounds at the progesterone receptor using a rabbit model. The McPhail test uses a scale of 0-4 with progesterone having the highest score of 4. RU486, on the other hand, has a score of less than 0.5 and is therefore considered a pure antagonist in this model. The McPhail test is described in Selye H., Textbook of Endocrinology, 1947, pp 345-346. Mcphail testing and in vivo characterization readily can be used to identify SPRMs. Using the McPhail test as a guide, SPRMs generally can be categorized as compounds having a McPhail score of between 0.5 and 3.5, more preferably between 0.5 and 3, and most preferably between 0.5 and 2. Compounds having such activities, as well as methods for synthesizing such compounds, have been described in U.S. Pat. Nos. 5,843,931; 5,519,027; 5,426,102; 5,244,886; 5,273,971; 5,446,063; 5,576,310 and 5,693,628; (all of which are herein incorporated by reference) as well as in PCT Patent Applications having publication numbers WO 01/26603; WO 01/34126; and WO 01/15679. Compounds that have previously been designated J867, J900, J956, J912, J914, and J1042 are all suitable for use in accordance with the methods provided herein. Such compounds include [4-[17β-Methoxy-17α-(methoxymethyl)-3-oxoestra-4,9-dien-11β-yl]benzaldehyd-(1E)-oxim]; [4-17β-Hydroxy-17α-(methoxymethyl)-3-oxoestra-4,9-dien-11β-yl]benzaldehyd-(1E)-oxim]; [4-17β-Methoxy-17α-(methoxymethyl)-3-oxoestra-4,9-dien-11β-yl]benzaldehyd-(1E)-[O-(ethoxy)carbonyl]oxim; [4-17β-Methoxy-17α-(methoxymethyl)-3-oxoestra-4,9-dien-11β-yl]benzaldehyd-(1E)-(O-acetyl)oxim]; and [4-[17β-Methoxy-17α-(methoxymethyl)-3-oxoestra-4,9-dien-11β-yl]benzaldehyd-(1E)- [O-(ethylamino)carbonyl)oxim].

SPRM's can be employed in methods to increase total testosterone levels as well as lower the levels of SHBG to thereby increase bioavailable (or free) testosterone. Advantageously, through the use of SPRMs, endogenous bioavailable testosterone levels are increased and therefore administration of exogenous testosterone is not necessary. Methods for increasing testosterone levels generally comprise administering a therapeutically effective amount of a SPRM to a patient in need of an increased testosterone level and/or decreased SHBG level.

A variety of conditions in men and women can benefit from an increase in testosterone levels. For example, in women, such conditions may include reduced libido (sex motivation, fantasy, and enjoyment), arousal, vaginal vasocongestion, mood changes, energy loss, vasomotor symptoms (hot flushes), depression, osteoporosis, and frailty. In men, for example, conditions related to reduced testosterone levels due to hypogonadism or andropause may include muscle wasting, frailty, osteoporosis, and anemia. As mentioned previously, increasing testosterone levels (more particularly free testosterone) and decreasing SHBG levels has been found to increase libido which is predominantly a psychological condition that is associated with sexual dysfumction in both men and women. Hence, a preferred method is administering a SPRM to a patient having sexual dysfunction. In cases where SPRMs are administered to a patient having sexual dysfunction, additional drugs may also be provided to alleviate physiological aspects of sexual dysfumction. For example, PDE5 inhibitors such as sildenafil, and vardenafil, as well as dopamine receptor agonists such as apomorphine have found utility for alleviating physiological aspects of sexual dysfunction such as, for example, erectile dysfunction and vaginal dryness. Hence, methods for treating sexual dysfunction are provided that comprise administering therapeutically effective amounts of a SPRM alone or in combination with a drug indicated for alleviating the physiological aspects of sexual dysfunction such as, for example, those mentioned above.

While providing SPRMs beneficially results in an increase of endogenous testosterone, there may be cases where patients may benefit from an additional administration of exogenous testosterone in addition to SPRM therapy. Hence, methods provided herein may also comprise administering testosterone to a patient in addition to a SPRM or in addition to a SPRM and a drug indicated for alleviating physiological aspects of sexual dysfunction.

The phrase “therapeutically effective amount” as used herein means a sufficient amount of, for example, a composition, compound, or formulation necessary to treat the desired disorder, at a reasonable benefit/risk ratio applicable to any medical treatment. As with other pharmaceuticals, it will be understood that the total daily usage of SPRMs or other drugs mentioned herein will be decided by a patient's attending physician within the scope of sound medical judgment. The specific therapeutically effective dose level for any particular patient will depend upon a variety of factors including the disorder being treated and the severity of the disorder; activity of the specific compound employed; the specific composition employed; the age, body weight, general health, sex and diet of the patient; the time administration, route of administration, and rate of excretion of the specific compound employed; the duration of the treatment; drugs used in combination or coincidental with the specific compound employed; and other factors known to those of ordinary skill in the medical arts. For example, it is well within the skill of the art to start doses of the compound at levels lower than required to achieve the desired therapeutic effect and to gradually increase the dosage until the desired effect is achieved. These parameters can then be employed to appropriately dose a particular patient such that a patient receives the desired effect.

Typically, the daily therapeutically effective amount of the compounds administered to a patient in single or divided doses range from about 0.1 to about 200 mg/kg body weight, more typically from about 0.25 to about 100 mg/kg body weight. Preferably, compounds are administered orally at doses between 0.5 mg/day and 500 mg/day, more preferably between 1 mg/day and 250 mg/day, and most preferably between 5 mg/day and 150 mg/day.

SPRMs, as well as other pharmaceutically acceptable compounds indicated for alleviating physiological aspects of sexual dysfunction, can be administered in a variety of forms. Compounds of this invention may be administered orally, ophthalmically, osmotically, parenterally (subcutaneously, intramuscularly, intrasternally, intravenously), rectally, topically, transdermally, or vaginally. Orally administered compounds in solid dosage forms may be administered as capsules, dragees, granules, pills, powders, and tablets. Ophthalmically and orally administered compounds in liquid dosage forms may be administered as elixirs, emulsions, microemulsions, solutions, suspensions, and syrups. Osmotically and topically administered compounds may be administered as creams, gels, inhalants, lotions, ointments, pastes, powders, solutions, and sprays. Parenterally administered compounds may be administered as aqueous or oleaginous solutions or aqueous or oleaginous suspensions, which suspensions comprise crystalline, amorphous, or otherwise insoluble forms of the compounds. Rectally and vaginally administered compounds may be administered as creams, gels, lotions, ointments, and pastes.

Depending upon the form of administration, SPRMs, as well as other compounds indicated for alleviating physiological aspects of sexual dysfumction, may be formulated or administered with or without a pharmaceutically acceptable excipient. Such excipients include encapsulating materials or formulation additives such as absorption accelerators, antioxidants, binders, buffers, coating agents, coloring agents, diluents, disintegrating agents, emulsifiers, extenders, fillers, flavoring agents, humectants, lubricants, perfumes, preservatives, propellants, releasing agents, sterilizing agents, sweeteners, solubilizers, wetting agents, and mixtures thereof.

For example, excipients for orally administered compounds in solid dosage forms include agar, alginic acid, aluminum hydroxide, benzyl alcohol, benzyl benzoate, 1,3-butylene glycol, castor oil, cellulose, cellulose acetate, cocoa butter, corn starch, corn oil, cottonseed oil, ethanol, ethyl acetate, ethyl carbonate, ethyl cellulose, ethyl laureate, ethyl oleate, gelatin, germ oil, glucose, glycerol, groundnut oil, isopropanol, isotonic saline, lactose, magnesium hydroxide, magnesium stearate, malt, olive oil, peanut oil, potassium phosphate salts, potato starch, propylene glycol, Ringer's solution, talc, tragacanth, water, safflower oil, sesame oil, sodium carboxymethyl cellulose, sodium lauryl sulfate, sodium phosphate salts, soybean oil, sucrose, tetrahydrofurfuryl alcohol, and mixtures thereof. Excipients for ophthalmically and orally administered compounds in liquid dosage forms include benzyl alcohol, benzyl benzoate, 1,3-butylene glycol, castor oil, corn oil, cottonseed oil, ethanol, ethyl acetate, ethyl carbonate, fatty acid esters of sorbitan, germ oil, groundnut oil, glycerol, isopropanol, olive oil, polyethylene glycols, propylene glycol, sesame oil, tetrahydrofurfuryl alcohol, water, and mixtures thereof. Excipients for osmotically administered compounds include chlorofluorohydrocarbons, ethanol, isopropanol, water, and mixtures thereof. Excipients for parenterally administered compounds include 1,3-butanediol, castor oil, corn oil, cottonseed oil, germ oil, groundnut oil, liposomes, oleic acid, olive oil, peanut oil, Ringer's solution, safflower oil, sesame oil, soybean oil, U.S.P. or isotonic sodium chloride solution, water, and mixtures thereof. Excipients for rectally and vaginally administered compounds include cocoa butter, polyethylene glycol, wax, and mixtures thereof.

The phrase “pharmaceutically acceptable” as used herein includes moieties or compounds that are, within the scope of sound medical judgment, suitable for use in contact with the tissues of humans and lower animals without undue toxicity, irritation, allergic response, and the like, and are commensurate with a reasonable benefit/risk ratio.

SPRMs and other pharmaceutically acceptable compounds indicated for alleviating physiological aspects of sexual dysfunction may separately be provided or packaged as kits. Advantageously, each compound of the kit may be packaged in per use groupings such that, for example, a daily prescription of each component can identified in order to enhance patient compliance. Sets of the compounds may be identified in a variety of ways. For example, a set of compounds may be identified on the package containing the compounds. Alternatively, external instructions may be provided with a set or sets of the compounds that, for example, identify a grouping and instruct a patient appropriate times to take the components of the kit. Convenience packs, such as those described above, are well known and take a variety of forms such as, for example, those described in U.S. Pat. Nos. 3,921,804; 4,964,539; 5,316,400; and 5,775,536. So-called “blister packs” are a common type of convenience pack and generally comprise a sheet of material that can be formed with blisters to contain a solid dosage form and a backing sheet sealed to the blistered material to maintain the dosage form in the individual blisters.

Alternatively, SPRMs and other pharmaceutically acceptable compounds indicated for alleviating physiological aspects of sexual dysfunction can be separately provided as a single dosage form. The dosage form employed is largely a matter of choice for those skilled in the art. Such dosage forms may be formulated with excipients exemplified above which also are a matter of choice that is dependent upon the particular dosage form employed.

The compounds and processes of this invention will be better understood in connection with the following examples.

### EXAMPLES

### Effects of SPRMs on Testosterone and SHBG Levels

The effect of SPRMs on testosterone levels and SHBG levels was studied using various doses of J867 over three months. The study was a phase II, multi-center, randomized, double blind, parallel group study of J867 at doses of 5 mg, 10 mg, and 25 mg once a day (QD)compared to placebo. Approximately 120 female subjects between the ages of 18 and 49 were enrolled in the study and randomly assigned to one of the four dosing groups above. During the 12 week study, testosterone and SHBG were measured at screening visits during weeks 2, 4, 8, and 12 of the study.

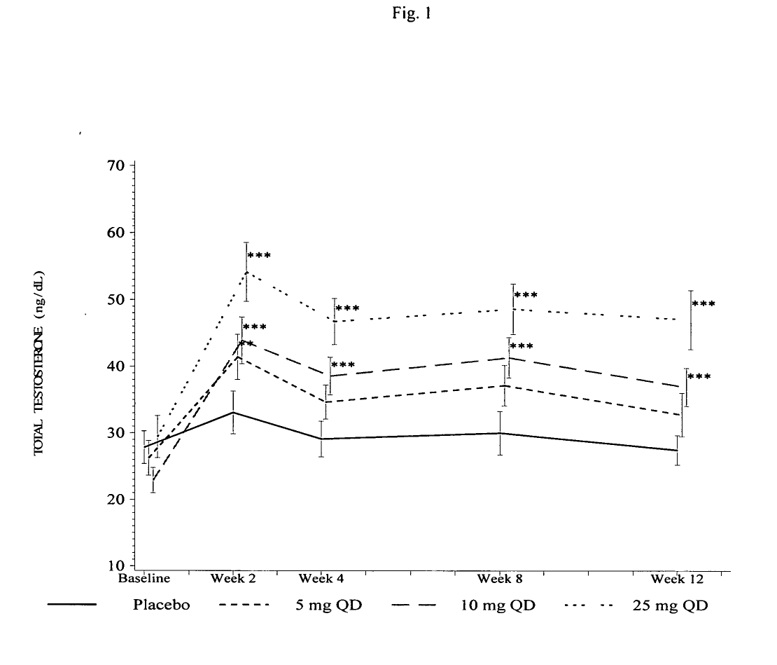

FIG. 1 is a graph showing total testosterone levels as a function of time during the study. As shown by FIG. 1, the study demonstrated that testosterone levels increased with all doses of the SPRM. Testosterone concentrations were relatively constant for the group receiving placebo. For all three groups receiving the SPRM, the largest mean increase of testosterone from baseline was observed at the week 2 visit. After this visit, a slight testosterone concentration decrease was observed, but testosterone concentrations were still elevated above baseline at the end of the study. Over the treatment period, the mean testosterone concentrations for the 5 mg QD group increased approximately 7-15 ng/dL; the mean increase for the 10 mg QD group was approximately 15-21 ng/dL; and the mean concentrations observed following treatment with 25 mg QD increased approximately 16-25 ng/dL.

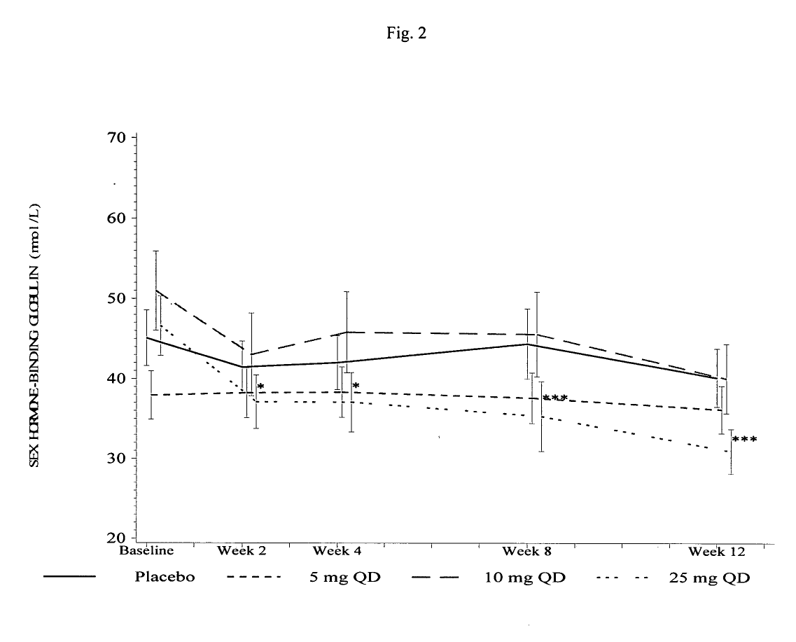

SHBG levels were also measured at weeks 2, 4, 8, and 12 of the study. The results of these measurements are shown in FIG. 2. As shown in FIG. 2, a decrease in SHBG during the treatment period of three months with the SPRM was observed.

The mean measurements of sex hormone-binding globulin (SHBG) during treatment with placebo group and the 5 mg QDgroup remained relatively constant. In the 10 mg QD and 25 mg QD groups, the mean level of SHBG during treatment decreased from baseline.

The foregoing examples are illustrative of the invention and are not intended to limit the same to the specifically disclosed compounds and processes. Variations and changes which are obvious to one skilled in the art are intended to be within the scope of this invention.

What is claimed is:

1. A method of treating sexual dysfunction comprising administering a therapeutically effective amount of a selective progesterone receptor modulator to a patient.

2. The method of claim 1 wherein the selective progesterone receptor modulator is selected from the group consisting of J867, J956.

3. The method of claim 1 wherein the patient is female.

4. The method of claim 1 wherein the patient is male.

5. The method of claim 1 further comprising administering to the patient a drug indicated for alleviating the physical symptoms associated with sexual dysfunction.

6. The method of claim 5 wherein the drug indicated for alleviating the physical symptoms associated with sexual dysfunction is a PDE5 inhibitor or a dopamine agonist.

7. A method for increasing testosterone levels in a patient comprising administering a therapeutically effective amount of a selective progesterone receptor modulator to a patient.

8. The method of claim 7 wherein the selective progesterone receptor modulator is selected from the group consisting of J867, J956.

9. The method of claim 7 wherein the patient is female.

10. The method of claim 7 wherein the patient is male.

11. A method for decreasing sex hormone binding globulin levels in a patient comprising administering a therapeutically effective amount of a selective progesterone receptor modulator to a patient.

12. The method of claim 11 wherein the selective progesterone receptor modulator is selected from the group consisting of J867 and J956.

13. The method of claim 11 wherein the patient is female.

14. The method of claim 11 wherein the patient is male.

15. A kit comprising a selective progesterone receptor modulator, and a drug indicated for alleviating the physical symptoms associated with sexual dysfunction.

16. A pharmaceutical formulation comprising a selective progesterone receptor modulator, and a drug indicated for alleviating the physical symptoms associated with sexual dysfunction.

17. A method of treating vaginal vasocongestion, mood changes, energy loss, hot flushes, depression, osteoporosis, hypogonadism, muscle wasting, anemia, and frailty comprising administering a therapeutically effective amount of a selective progesterone receptor modulator to a patient.
